(19) United States
(12) Patent Application Publication
(10) Pub. No.: US 2008/0220170 A1
(43) Pub. Date: Sep. 11, 2008

(54) Use of Alkoxylated Amines to Improve Water Repellency

(76) Inventor: Mark Arthur Josepha Van Der Flaas, Beerse (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/065,198

(22) PCT Filed: Sep. 1, 2006
(86) PCT No.: PCT/EP2006/065901

§ 371 Date: Feb. 28, 2008

(30) Foreign Application Priority Data
Sep. 1, 2005 (EP) 05108018.2

(51) Int. Cl. B05D 3/00; B05D 1/04; C07C 211/01

(52) U.S. Cl. 427/297; 427/458; 564/463

(57) ABSTRACT

The present invention relates to the use of alkoxylated amines for treating wood and other cellulose materials in order to enhance their water repellent properties and to reduce water uptake when treated wood or other cellulose materials are brought into contact with water.

The present invention relates to the use of alkoxylated amines for treating wood and other cellulose materials in order to enhance their water repellent properties and to reduce water uptake when treated wood or other cellulose materials are brought into contact with water.

As soon as trees are cut the wood starts to decay by fungal attack. One of the most successful ways to protect wood against decay is to impregnate the wood by means of a pressure process using a water-borne wood preservative. Such treatments can be made more effective by incorporating into the treatment process an additive which will improve the dimensional stability of the treated wood by imparting a high degree of water repellency.

The object of water repellent treatment of wood is to reduce the wettability of the wood surface so that liquid water does not form a coherent film and to reduce the absorption of liquid water. This prevention of the absorption of liquid water gives a degree of dimensional stability and prevents rapid swelling and shrinkage during wetting and drying and is also effective in reducing the rate of mechanical degradation, surface checking and cracking in treated wood during initial drying or in service. The natural affinity of wood for water is reduced by treatment with water repellents thereby minimizing wood splitting, end checking and grain raising resulting in an increase of the service life of wood and finish.

Wood can be made water repellent by treating it with rosin solutions, coating compositions containing latex or wax emulsions, or hydrocarbon emulsions comprising surfactants.

Alkoxylated amines of the general formula (I), also known as ethomeens and ethoduomeens, are cationic surfactants used in a wide variety of applications such as viscose production, feed additives, agro additives applications, cleaning, flotation agents in mining, emulsifiers and adhesion promoters for bitumen, and anti-caking agents for fertilisers. WO-96/10332, WO-03/065807 and EP-1,273,233 disclose these alkoxylated amines for enhancing the fungicidal activity of copper cations, triazoles and oxathiazines, respectively.

It has now been found that alkoxylated amines of formula (I) can be used as water repellents to impregnate wood and improve its water repellency and reduce the uptake of water when treated wood is brought into contact with water.

The alkoxylated amines of formula (I) have the following general formula

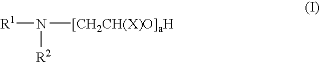

wherein
R¹ is a C₈₋₂₀alkyl;

R² is —[CH₂CH(X)O]_{b}H (a-1)

or

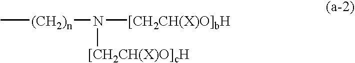

n is an integer from 1 to 4;
each a, b, and c independently are integers which can be 1 to 20;
each X independently is selected from the group consisting of hydrogen, methyl, ethyl, and phenyl.

A group of interesting compounds of formula (I) are those compounds of formula (I) wherein each a, b, and c independently are integers which can be 1 to 6.

A first particular group of alkoxylated amines of formula (I) are those compounds of formula (I) wherein R² represents radical (a-1).

A second particular group of alkoxylated amines of formula (I) are those compounds of formula (I) wherein R² represents radical (a-2).

More particular alkoxylated amines of formula (I) are those alkoxylated amines of formula (I) wherein one or more of the following restrictions apply

a) n is an integer 2 or 3, preferably n is 3;
b) X is hydrogen;
c) R is a C₁₀₋₂₀alkyl, preferably cocoalkyl or tallowalkyl.

Commercially available alkoxylated amines of formula (I) wherein R² represents radical (a-1) are listed in the following table

| Product name | Chemical name |
|---|---|
| Ethomeen C/12 | bis-(2-hydroxyethyl)cocoalkylamine |
| Ethomeen C/15 | ethoxylated(5)cocoalkylamine |
| Ethomeen C/25 | ethoxylated(15)cocoalkylamine |
| Ethomeen O/12 | bis-(2-hydroxyethyl)oleylamine |
| Ethomeen O/17 | ethoxylated(7)oleylamine |
| Ethomeen O/20 | ethoxylated(10)oleylamine |
| Ethomeen S/15 | ethoxylated(5)soyaalkylamine |
| Ethomeen S/25 | ethoxylated(15)soyaalkylamine |
| Ethomeen T/12 | bis-(2-hydroxyethyl)tallowalkylamine |
| Ethomeen T/15 | ethoxylated(5)tallowalkylamine |
| Ethomeen T/25 | ethoxylated(15)tallowalkylamine |
| Ethomeen HT/12 | bis-(2-hydroxyethyl) hydrogenatedtallowalkylamine |
| Ethomeen HT/14 | ethoxylated(4) hydrogenatedtallowalkylamine |
| Ethomeen HT/17 | ethoxylated(7) hydrogenatedtallowalkylamine |
| Ethomeen HT/20 | ethoxylated(10) hydrogenatedtallowalkylamine |
| Ethomeen HT/25 | ethoxylated(15) hydrogenatedtallowalkylamine |
| Ethomeen HT/30 | ethoxylated(20) hydrogenatedtallowalkylamine |
| Ethomeen 12/12 | bis-(2-hydroxyethyl)dodecylamine |
| Ethomeen 18/12 | bis-(2-hydroxyethyl)octadecylamine |

Commercially available alkoxylated amines of formula (I) wherein R² represents radical (a-2) are listed in the following table

| Product name | Chemical name |
|---|---|
| Propoduomeen C13 | N,N′,N′-tris(2-hydroxypropyl)-N-cocoalkyl-1,3- |
| | diaminopropane |
| Ethoduomeen T11 | mono-(2-hydroxyethyl)-N-tallowalkyl-1,3- |
| | diaminopropane |
| Ethoduomeen T13 | N,N′,N′-tris-(2-hydroxyethyl)-N-tallowalkyl-1,3- |
| | diamino-propane |
| Ethoduomeen T25 | N,N′,N′-polyoxyethylene(15)-N-tallowalkyl-1,3- |
| | diamino-propane |
| Ethoduomeen C13 | N,N′,N′-tris(2-hydroxyethyl)-N-cocoalkyl-1,3- |
| | diaminopropane |

Most preferred alkoxylated amines of formula (I) are N,N′,N′-tris(2-hydroxyethyl)-N-cocoalkyl-1,3-diaminopropane (=Ethoduomeen T13) and N,N′,N′-tris(2-hydroxyethyl)-N-cocoalkyl-1,3-diaminopropane (=Ethoduomeen C13).

As used herein, “wood,” “wood material” and “wood products” shall mean all forms of wood, for example, solid wood (such as timber or lumber in the form of logs, beams, planks, sheets and boards), wood composite materials (such as wood fiber board, chip board and particle board) and all products made from wood and wood-composite materials (such as mill frames, decking, siding, siding cladding, roof shingles, utility poles, and railway sleepers).

In a further object of the invention the alkoxylated amines of formula (I) can be used to improve the water repellency of various cellulose materials such as paper and cardboard.

The alkoxylated amines of formula (I) are used in the form of a liquid solution for treating wood in order to enhance the water repellent properties of wood or for reducing water uptake when treated wood is brought into contact with water. However the alkoxylated amines of formula (I) may also be applied neat to the wood.

The alkoxylated amines of formula (I) can be used in the form of a liquid composition which can be a ready for use composition or a concentrated composition that has to be diluted shortly before use. These liquid compositions comprise the alkoxylated amines of formula (I) in an amount ranging from 0.1 to 90% by weight.

Appropriate carriers for liquid compositions comprising the water repellent alkoxylated amines of formula (I) are any liquid that does not adversely affect the alkoxylated amines of formula (I), for example, water, alcohols (for example, methyl alcohol, ethyl alcohol, ethylene glycol, propylene glycol, diethylene glycol, glycerin, etc.), ketones (for example, acetone, methyl ethyl ketone, etc.), ethers (for example, dioxane, tetrahydrofurane, cellosolve, diethylene glycol dimethyl ether, etc.), aliphatic hydrocarbons (for example, hexane, kerosene, etc.), aromatic hydrocarbons (for example, benzene, toluene, xylene, solvent naphtha, methyl naphthalene, etc.), halogenated hydrocarbons (for example, chloroform, carbon tetrachloride, etc.), acid amides (for example, dimethyl formamide, etc.), esters (for example, methyl acetate ester, ethyl acetate ester, butyl acetate ester, fatty acid glycerin ester, etc.), and nitriles (for example, acetonitrile, etc.). These solvents may be used either singly or in combination of two or more species.

The carrier for the liquid compositions can also be supercritical CO₂.

The alkoxylated amines of formula (I) can also be used as a water repellent additive in compositions for wood treatment that are used in the first instance for making wood more resistant to attack by bacteria, fungi, molds, or insects.

The liquid compositions comprising the alkoxylated amines of formula (I) can be applied to wood by any known technique, for example by dipping, spraying, electrostatic spraying, curtain coating, brush coating, dip coating, flow coating, roll coating and vacuum/pressure treatment methods which utilize pressure difference for penetration of the liquid.

### EXPERIMENTAL PART

### Materials and Methods

### Treatment of Wood

| | |
|---|---|
| Alkoxylated amines: | Ethoduomeen C/13 (Akzo Nobel), CAS 90367-21-8. |
| | Ethoduomeen T/13 (Akzo Nobel), CAS 90367-27-4. |
| Test concentrations: | 500, 2500 and 12500 mg/litre (ppm) in demineralised water, |
| | however ethoduomeen T/13 at 12500 ppm was dissolved in |
| | 0.2% 0.1N HCl (= 2.10⁻⁴ M or 7.3 ppm) |
| Solvent: | sterile demineralised water |
| Species of wood: | Scots Pine (Pinus sylvestris); 556.58 kg/m³ |
| Wood dimensions: | 15 × 25 × 50 mm (volume 18.75 cm³, mean laboratory dry weight |
| | of blocks (n = 110) 10.50 g, with standard deviation 0.72) |
| Treatment: | pressure treatment: 400 mbar for 10 minutes |
| | surface application: dipping during 30 seconds |
| Control: | treatment with demineralised water, or |
| | treatment with demineralised water with 0.2% 0.1N HCl added |
| | (= 2.10⁻⁴ M or 7.3 ppm) (only for the ethoduomeen T/13 at |
| | 12500 ppm test solution) |
| Replicates: | 5 |
| Fixation: | 14 days |
| Drying: | 14 days + 3 days |

### Water Repellency Test

Evaluation method: % weight increase after dipping in demineralised water for 30 minutes at room temperature
   % tangential swelling as measured along the 25 mm side of the blocks after dipping in demineralised water for 30 minutes at room temperature (AWPA standard E4-78)
Statistics: Treatments were compared to controls using an ANOVA with Dunnett's correction for multiple comparisons. For an overall p=0.05, critical t-value=2.72 (g=6, df=32), resulting in α=0.0105.

### Results and Discussion

TABLE 1
% weight increase after 30 minutes dipping of wood treated with
ethoduomeen C/13 and T/13

| | | | Ethoduomeen | | | Ethoduomeen | | |
|---|---|---|---|---|---|---|---|---|
| | Controls | | T/13 (ppm) | | | C/13 (ppm) | | |
| | water | HCl | 500 | 2500 | 12500 | 500 | 2500 | 12500 |
| Vacuum | 44.15 | 44.67 | 43.28 | 22.40 | 21.61 | 33.17 | 23.23 | 27.97 |
| | 46.42 | 43.60 | 42.06 | 20.48 | 17.35 | 31.80 | 38.69 | 24.79 |
| | 49.85 | 46.99 | 39.20 | 25.56 | 17.52 | 31.29 | 23.13 | 21.22 |
| | 46.27 | 37.93 | 41.59 | 32.74 | 24.13 | 35.77 | 32.81 | 37.78 |
| | 47.57 | 46.69 | 38.56 | 25.97 | 18.93 | 44.79 | 27.67 | 22.00 |
| Means* | 46.85 | 43.98 | 40.94 | 25.43* | 19.91* | 35.37* | 29.10* | 26.75* |
| Dipping | 31.07 | 37.23 | 35.53 | 32.18 | 17.86 | 33.11 | 25.61 | 17.71 |
| | 33.83 | 25.32 | 28.76 | 31.53 | 23.41 | 22.86 | 28.68 | 22.04 |
| | 28.90 | 39.15 | 37.75 | 34.44 | 24.45 | 32.69 | 31.76 | 22.83 |
| | 43.18 | 20.68 | 37.16 | 26.05 | 24.46 | 32.21 | 27.03 | 26.99 |
| | 31.42 | 27.59 | 30.05 | 39.83 | 22.12 | 35.16 | 17.75 | 23.08 |
| Means* | 33.68 | 29.99 | 33.85 | 32.81 | 22.46* | 31.21 | 26.17 | 22.53* |

Means indicated by * are significantly different (P = 0.05) from control.

After vacuum infiltration, the mean % weight increase by water uptake of treated blocks is significantly lower than controls (5% level) for all tested concentrations of ethoduomeen C/13 and for 2500 and 12500 ppm ethoduomeen T/13.

After dipping, the mean % weight increase by water uptake of treated blocks is significantly lower than controls (5% level) for 12500 ppm ethoduomeen C/13 and T/13.

TABLE 2
% tangential swelling after 30 minutes dipping of wood treated with
ethoduomeen C/13 and T/13

| | | | Ethoduomeen | | | Ethoduomeen | | |
|---|---|---|---|---|---|---|---|---|
| | Controls | | T/13 (ppm) | | | C/13 (ppm) | | |
| | water | HCl | 500 | 2500 | 12500 | 500 | 2500 | 12500 |
| Vacuum | 3.76 | 3.16 | 3.29 | 0.84 | 1.80 | 2.50 | 0.92 | 2.92 |
| | 4.08 | 3.55 | 3.05 | 2.00 | 1.21 | 2.72 | 3.72 | 1.43 |
| | 3.63 | 2.83 | 3.81 | 2.53 | 1.52 | 1.39 | 1.93 | 2.22 |
| | 4.76 | 3.48 | 3.68 | 2.51 | 1.40 | 1.98 | 2.56 | 3.12 |
| | 4.80 | 3.44 | 2.90 | 1.36 | 1.56 | 3.30 | 1.28 | 0.48 |
| Means* | 4.21 | 3.29 | 3.35* | 1.85* | 1.50* | 2.38* | 2.08* | 2.03* |
| Dipping | 2.64 | 1.97 | 2.08 | 2.02 | 0.80 | 1.45 | 1.20 | 1.58 |
| | 2.21 | 1.40 | 2.40 | 1.52 | 1.17 | 0.56 | 1.41 | 1.21 |
| | 1.81 | 1.81 | 1.93 | 2.38 | 0.97 | 2.10 | 1.52 | 1.20 |
| | 3.66 | 0.89 | 1.93 | 0.68 | 0.79 | 1.40 | 1.68 | 1.46 |
| | 1.80 | 1.45 | 1.41 | 2.46 | 0.88 | 1.96 | 1.08 | 0.84 |
| Means* | 2.42 | 1.50 | 1.95 | 1.81 | 0.92* | 1.49 | 1.38 | 1.26* |

Means indicated by * are significantly different (P = 0.05) from control. After vacuum infiltration, the mean % tangential swelling as a result of water uptake of treated blocks is significantly lower than controls (5% level) for all tested concentrations of Ethoduomeen C/13 and T/13.

After dipping, the mean % tangential swelling as a result of water uptake of treated blocks is significantly lower than controls (5% level) for 12500 ppm ethoduomeen C/13 and T/13.

### CONCLUSION

Vacuum infiltration of pine wood with the alkoxylated amines ethoduomeen C/13 and T/13 significantly diminishes water uptake of the treated wood, even at relatively low concentrations of the amines.
Brief dipping of pine wood in ethoduomeen solutions only gives a significant reduction of water uptake when a high concentration of ethoduomeen C/13 or T/13 is used.

1. A method for improving the water repellency of wood or reducing the amount of water uptake by wood, comprising treating the wood with alkoxylated amines of formula 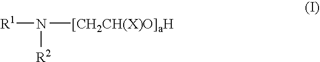
wherein
R¹ is a C₈₋₂₀alkyl;
R^{2 is —[CH}₂CH(X)O]_{b}H (a-1)
or 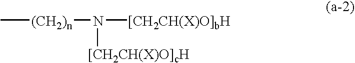
n is an integer from 1 to 4;
each a, b, and c independently are integers which can be 1 to 20;
each X independently is selected from the group consisting of hydrogen, methyl, ethyl, and phenyl.

2. The method according to claim 1 wherein R² is a radical formula (a-1).

3. The method according to claim 1 wherein R² is a radical formula (a-2).

4. The method according to claim 1 wherein the alkoxylated amine of formula (I) is N,N′,N′-tris(2-hydroxyethyl)-N-cocoalkyl-1,3-diamino-propane.

5. The method according to claim 1 wherein the alkoxylated amine of formula (I) is N,N′,N′-tris-(2-hydroxyethyl)-N-tallowalkyl-1,3-diaminopropane.

6. The method according to claim 1 wherein the wood is treated with a composition comprising alkoxylated amines of formula (I) as defined in claim 1.

7. The method according to claim 6 wherein treatment comprises vacuum or pressure treatment of wood with a liquid composition comprising alkoxylated amines of formula (I) as defined in claim 1.

8. The method according to claim 7 wherein treatment comprises dipping, spraying, electrostatic spraying, curtain coating, brush coating, dip coating, flow coating, or roll coating of wood with a liquid composition comprising an alkoxylated amines of formula (I) as defined in claim 1.

9. The method according to claim 7 wherein the alkoxylated amines of formula (I) are present in the liquid composition in an amount ranging from 0.1 to 90% by weight.

10. The method according to claim 8 wherein the alkoxylated amines of formula (I) are present in the liquid composition in an amount ranging from 0.1 to 90% by weight.
